(19) United States
(12) Patent Application Publication
(10) Pub. No.: US 2022/0325323 A1
(43) Pub. Date: Oct. 13, 2022

(54) SKIN MICROBIOME MONITORING FOR IMPROVING SYSTEMIC HEALTH

(71) Applicant: MY-OR DIAGNOSTICS LTD., Zikhron Yaakov (IL)

(72) Inventors: Ron KOHN, Kefar Uriya (IL); Michael BRANDWEIN, Elazar (IL); Ariel KATZ, Zichron Yaakov (IL)

(21) Appl. No.: 17/639,399

(22) PCT Filed: Sep. 2, 2020
(86) PCT No.: PCT/IL2020/050955

§ 371 Date: Mar. 1, 2022

Related U.S. Application Data
(60) Provisional application No. 62/894,895, filed on Sep. 2, 2019.

(51) Int. Cl. C12Q 1/689; A23L 33/20

(52) U.S. Cl. CPC C12Q 1/689; A23L 33/20

(57) ABSTRACT

The present invention is directed to a method for determining predisposition to developing a metabolic syndrome or a condition associated therewith in a subject including determining the bacterial diversity in a facial skin of the subject. Further provided is a method for treating or preventing a metabolic syndrome or a condition associated therewith in a subject in need thereof.

### CROSS-REFERENCE TO RELATED APPLICATIONS

This application claims the benefit of priority of U.S. Provisional Patent Application No. 62/894,895 titled “SKIN MICROBIOME MONITORING FOR IMPROVING SYSTEMIC HEALTH”, filed Sep. 2, 2019, the contents of which are incorporated herein by reference in their entirety.

### FIELD OF THE INVENTION

The invention relates generally to the field of microbiome and diseases associated therewith.

### BACKGROUND

The global obesity pandemic has far-reaching social, economic and health-related ramifications. Its incidence has risen at an alarming rate, with nearly one-fifth of children and over one-third of adults in the US are obese, and national initiatives and/or programs to curb obesity have so far been unsuccessful. Obesity is the result of higher energy intake than expenditure and is the central factor leading to the development of the metabolic syndrome. Several important mediators have been identified in obesity, including the Western diet, oxidative stress, the microbiome, and chronic inflammation.

Early microbiome studies established the link between obesity and the human gut microbiome. These studies have shown that the ratio of Firmicutes to Bacteroidetes is elevated in both obese mice and humans. Furthermore, weight loss induces a compositional shift whereby Bacteroidetes increase and Firmicutes decrease. Further studies documented the lack of microbial diversity of the Western gut microbiome compared to populations which consume a more traditional plant-based diet.

For the most part, microbiome studies on obesity have been limited to the gut microbiome.

The American Gut Project (AGP), launched in 2012, aims to establish a comprehensive reference set for microbiome research by engaging the general public globally. It allows members of the general public to submit oral, skin or fecal samples for microbiome profiling and the results are then made available to the donor and are deidentified and added to the larger, freely-available database. This paradigm-shifting endeavor has shown that the diversity of plants that are consumed are strongly correlated with gut microbiome composition.

A method for detecting or diagnosing metabolic syndrome or a condition associated therewith based on the subject's skin microbiome, is still greatly needed.

### SUMMARY

The following embodiments and aspects thereof are described and illustrated in conjunction with systems, tools and methods which are meant to be exemplary and illustrative, not limiting in scope.

According to a first aspect, there is provided a method for determining predisposition to developing a metabolic syndrome or a condition associated therewith in a subject, the method comprising: determining the bacterial diversity in a facial skin of the subject; and comparing the determined bacterial diversity to a control, thereby determining predisposition to developing a metabolic syndrome in the subject.

According to another aspect, there is provided a method for treating or preventing a metabolic syndrome or a condition associated therewith in a subject in need thereof, the method comprising: (a) determining whether the subject is at increased risk of developing a metabolic syndrome or a condition associated therewith, according to the method disclosed herein; and administering to a subject determined as being at an increased risk, a therapeutically effective amount of a pharmaceutical or a nutraceutical composition comprising an agent selected from the group consisting of: an appetite suppressant, a probiotic agent, a prebiotic agent, an anti-inflammatory drug, a topical solution, and any combination thereof, thereby treating or preventing a metabolic syndrome or a condition associated therewith in a subject.

In some embodiments, determining comprises determining any one of: abundance of Corynebacteriaceae species, ratio of Corynebacteriaceae to Staphylococcaceae, and both, in a facial skin of the subject.

In some embodiments, a reduction of at least 5% in bacterial diversity in a facial skin of the subject compared to control is indicative of the subject being at increased risk of developing a metabolic syndrome or a condition associated therewith.

In some embodiments, an abundance of at least 15% of a Corynebacteriaceae species in a facial skin of the subject is indicative of the subject being at increased risk of developing a metabolic syndrome or a condition associated therewith.

In some embodiments, a Corynebacteriaceae to Staphylococcaceae abundance ratio ranging from 3.2:1 to 9:1 in a facial skin of the subject is indicative of the subject being at increased risk of developing a metabolic syndrome or a condition associated therewith.

In some embodiments, the subject has a body mass index (BMI) value ranging from 19 to 29.

In some embodiments, the subject has a BMI value ranging from 19 to 26.

In some embodiments, facial skin is the skin of any one of: the forehead, the glabella, or a combination thereof.

In some embodiments, the metabolic syndrome or the condition associated therewith is selected from the group consisting of: obesity, pre-diabetes, diabetes, hyperglycemia, diabetic dyslipidemia, hyperlipidemia, hypertriglyceridemia, hypercholesterolemia, hyperinsulinemia, and insulin-resistance or insulin-resistance related.

In some embodiments, determining is determining in a sample derived from the subject.

In some embodiments, the method further comprises providing a sample from the subject and performing the determining in the sample.

In some embodiments, the method further comprises modifying the diet of the subject.

In some embodiments, modifying is providing any one of: a diet low on calories, a diet low on fat content, a diet low on carbohydrates content, and any combination thereof.

In some embodiments, modifying is providing a diet low on calories.

In some embodiments, modifying is alternating the timing of food consumption.

In some embodiments, preventing comprises any one of: reducing the severity, delaying the onset, reducing the cumulative incidence, and any combination thereof, of the metabolic syndrome or the condition associated therewith.

In some embodiments, the metabolic syndrome or the condition associated therewith is selected from the group consisting of: obesity, pre-diabetes, diabetes, hyperglycemia, diabetic dyslipidemia, hyperlipidemia, hypertriglyceridemia, hypercholesterolemia, hyperinsulinemia, and insulin-resistance or insulin-resistance related.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

In addition to the exemplary aspects and embodiments described above, further aspects and embodiments will become apparent by reference to the figures and by study of the following detailed description.

### BRIEF DESCRIPTION OF THE FIGURES

FIG. 1 includes a graph showing clustering of microbial communities by body site. Gut, oral and skin sites cluster separately, thereby necessitating research hypothesis that look at these communities separately.

FIGS. 2A-2C include donut charts and vertical bar graphs showing taxonomic composition of microbial communities by body site. Gut, oral and skin sites are all dominated by Firmicutes, Bacteroidetes, Proteobacteria and Actinobacteria (2A), yet their percent community composition is significantly different between sites (2B-2C). Gut, oral and skin body sites are inhabited by varying bacterial families, with Bacteroidaceae, Ruminococcaceae and Lachnospiraceae dominating gut samples, Streptococcaceae and Micrococcaceae dominating oral samples and Staphylococcaceae and Corynebacteriaceae dominating skin samples.

FIGS. 3A-3C include graphs showing bacterial community diversity according to body site and BMI category. Gut (3A), oral (3B), and skin (3C) Shannon diversity measurements are presented according to BMI category. Gut microbial diversity is significantly different between all three categories, while no significance is observed between oral communities. There is a significant difference in microbial diversity between underweight and normal individuals and between underweight and obese individuals in skin communities.

FIG. 4 includes a graph showing the clustering of skin microbial communities by BMI category. Skin microbial communities are partitioned by BMI, with underweight individuals' skin microbiome markedly different than those of overweight and obese individuals. Normal individuals are plotted, yet their points are smaller to allow visualization of the other groups.

FIGS. 5A-5B include graphs showing differentially abundant bacteria by BMI category. LEfSe results indicating significantly enriched taxa by BMI category (5A). Additionally, Corynebacterium relative abundance correlates significantly with BMI (5B, p=0.0002). Point size is reflective of sample size in BMI levels portrayed.

FIGS. 6A-6C include donut charts showing microbiome taxonomy in the gut (6A), oral (6B), and skin (6C). BMI categories (overweight/obese, normal, and underweight) are indicated.

FIGS. 7A-7E include graphs and pie charts showing taxonomical composition of skin microbiome samples by skin site. (7A and 7B) are graphs showing phyla and families distribution, respectively. (7A) shows that Actinobacteria, in contrast to Proteobacteria, were more prominent in nares than in the palm and in the forehead. (7C) is a graph showing nare clustering separately from forehead and palm in PCoA (beta diversity). (7D and 7E) are graphs of Shannon diversity (7D) and Faith's phylogenetic diversity (7E) representing alpha diversity measures, showing statistically significant differences between skin sites.

FIGS. 8A-8D include graphs showing forehead microbiome analyses. (8A) is a Shannon (alpha) diversity analysis of forehead samples by BMI category. Statistical significance is shown between groups. (8B) is a graph showing that bacterial populations derived from forehead skin of underweight subjects clustered separately from overweight and obese. (8C) is a graph showing statistically significant correlation between Corynebacterium relative abundance and BMI. (8D) is a LDA analysis with list of bacteria over-/under-represented in obese/overweight or underweight individuals.

FIGS. 9A-9C include graphs showing nare microbiome analyses. (9A) is a Shannon (alpha) diversity analysis of nare samples by BMI category. Statistical significance is not shown between groups. (9B) is a graph showing that bacterial populations derived from nare skin of underweight subjects clustered separately from overweight and obese. (9C) is a graph showing statistically significant correlation between Corynebacterium relative abundance and BMI.

FIGS. 10A-10C include graphs showing palm microbiome analyses. (10A) is a Shannon (alpha) diversity analysis of palm skin samples by BMI category. Statistical significance is not shown between groups. (10B) is a graph showing that bacterial populations derived from palm skin of underweight subjects did not cluster separately from overweight and obese. (10C) is a graph showing statistically significant correlation between Corynebacterium relative abundance and BMI.

### DETAILED DESCRIPTION

According to some embodiments, there is provided a method for determining predisposition to developing a metabolic syndrome or a condition associated therewith in a subject, comprising determining the bacterial diversity in a facial skin of the subject; and comparing the determined bacterial diversity to a control, thereby determining predisposition to developing a metabolic syndrome in the subject.

According to some embodiments, there is provided a method for treating or preventing a metabolic syndrome or a condition associated therewith in a subject in need thereof, the method comprising: determining whether the subject is at increased risk of developing a metabolic syndrome or a condition associated therewith; and administering to a subject determined as being at an increased risk, a therapeutically effective amount of a composition comprising an agent selected from: an appetite suppressant, a probiotic agent, a prebiotic agent, an anti-inflammatory drug, a topical solution, or any combination thereof.

As used herein, the term “predisposition” refers to the susceptibility of a subject to a syndrome, a disease, or a condition associated therewith such as a metabolic syndrome. In some embodiments, determining a predisposition comprises determining the presence of the disease itself. In some embodiments, determining a predisposition comprises any one of: determining the risk of developing the disease, determining the susceptibility of the subject to developing the disease, having a poor prognosis for the disease, or any combination thereof. In some embodiments, a subject having a predisposition to a disease is at risk or at increased risk of developing the disease.

The terms “predisposition” and “likelihood” are used herein interchangeably.

In some embodiments, the method is directed to determining predisposition to developing a metabolic syndrome or a condition associated therewith, based on a skin microbiome analysis.

As used herein, the term “skin microbiome” refers to the microbiome of a facial skin. In some embodiments, facial skin comprises the skin of the forehead, the skin of the glabella, or any combination thereof.

In some embodiments, skin microbiome analysis comprises the analysis of facial skin microbiome. In some embodiments, facial skin comprises the skin of any part of a subject's face. In some embodiments, facial skin comprises the skin of the forehead skin, the skin of the glabella, or any combination thereof.

In some embodiments, skin microbiome analysis comprises determining any one of: abundance of a microorganism species or a strain thereof, the ratio of two or more species or strains of microorganisms, or both, in facial skin or any sample obtained or derived therefrom.

In one embodiment, a skin microbiome indicative of increased likelihood to develop a metabolic syndrome or a condition associated therewith comprises reduced bacterial community diversity, e.g., reduced number of different bacterial species, strains, or both. In one embodiment, determining that a skin microbiome is indicative of increased likelihood to develop a metabolic syndrome or a condition associated therewith comprises determining any one of: abundance of a species belonging to any family selected from: Streptococcaceae, Corynebacteriaceae, Staphylococcaceae, Micrococcaceae, Neisseriaceae, Pasteurellaceae, Prevotellaceae, and Moraxellaceae, ratio of two or more species belonging to any one of the aforementioned families, or both, in a facial skin of said subject. In another embodiment, a skin microbiome indicative of increased likelihood to develop the metabolic syndrome or a condition associated therewith comprises colonization of one or more bacteria belonging to any family selected from: Streptococcaceae, Corynebacteriaceae, Staphylococcaceae, Micrococcaceae, Neisseriaceae, Pasteurellaceae, Prevotellaceae, and Moraxellaceae. In another embodiment, a skin microbiome indicative of increased likelihood to develop the metabolic syndrome or a condition associated therewith comprises Corynebacterium colonization. In another embodiment, a skin microbiome indicative of increased likelihood to develop the metabolic syndrome or a condition associated therewith comprises Staphylococcus aureus colonization. In another embodiment, a skin microbiome indicative of increased likelihood to develop the metabolic syndrome or a condition associated therewith comprises high Corynebacterium kroppenstedtii colonization. In another embodiment, a skin microbiome indicative of increased likelihood to develop the metabolic syndrome or a condition associated therewith comprises high Staphylococcus aureus colonization. In another embodiment, a skin microbiome indicative of increased likelihood to develop the metabolic syndrome or a condition associated therewith comprises increased Corynebacterium, e.g., C. kroppenstedtii, colonization, increased Staphylococcus, e.g., S. aureus, colonization, reduced S. epidermidis colonization, reduced S. hominis colonization, or any combination thereof.

Methods of determining bacterial diversity are common and would be apparent to one of ordinary skill in the art. Non-limiting examples of methods for determining bacterial diversity, such as exemplified hereinbelow, include but are not limited to, phylogenetic diversity (PD)-whole-tree, e.g., Shannon diversity, and Bray-Curtis dissimilarity. Alpha diversity and taxonomic relative abundance can be further compared with the Wilcoxon rank sum test in R, and multiple hypothesis correction can be carried out using a false discovery rate.

In some embodiments, the method comprises determining the bacterial diversity prior to the appearance of the metabolic syndrome or a condition associated therewith. In some embodiments, the method comprises determining cutaneous bacterial diversity in a normally appearing skin.

As used herein, the term “normally appearing” refers to the gross morphology of a skin tissue as observed by the naked eye, by microscopy, or both.

In some embodiments, the determining excludes determining bacterial diversity on a cutaneous lesion evident after the appearance of the metabolic syndrome or a condition associated therewith. In some embodiments, the determining excludes determining bacterial diversity on a cutaneous lesion known to be induced by the metabolic syndrome.

As used herein, the terms “high”, “low”, “increased”, and “decreased” are compared to control. In some embodiments, the control is a skin microbiome of a healthy subject. In some embodiments, the control is a skin microbiome of a non-facial skin derived or obtained from either the subject or a healthy subject. In some embodiments, the control is a microbiome of a non-skin tissue derived or obtained from either the subject or a healthy subject. In some embodiments, the control is value threshold representing a healthy subject or a skin microbiome of same.

In some embodiments, the method of the invention is directed to a skin microbiome analysis comprising determining any one of: the abundance of a Corynebacteriaceae species or a strain thereof, the ratio of a Corynebacteriaceae species or a strain thereof to a Staphylococcaceae species or a strain thereof, or both.

In some embodiments, determining the abundance of any one of Corynebacteriaceae species, Staphylococcaceae species, or both, comprises DNA sequencing a skin microbiome for or using Corynebacteriaceae-specific sequence, Staphylococcaceae-specific sequences, or both.

In some embodiments, the method comprises a determining step in a sample derived from the subject. In some embodiments, the determining step is performed in vitro.

As used herein, in vitro is in a tube, a plate, or any equivalent thereof. In some embodiments, in vitro is outside a subject's body. In some embodiments, in vitro comprises ex vivo.

In some embodiments, the method further comprises providing a sample obtained, isolated, or derived from the subject, and performing the determining step in the sample.

In one embodiment, the step of detecting Staphylococcus colonization comprises detecting S. aureus, S. epidermidis, S. hominis, or a combination thereof. In one embodiment, the step of detecting Staphylococcus colonization on the skin of the subject comprises identification of a specific nucleotide sequence unique to cutaneous associated S. aureus species via PCR amplification of a gene fragment by gene-specific PCR primers.

In one embodiment, a step of analyzing the microbiome comprises culturing the sample derived from the skin of the subject on selective substrate, for example agar. In another embodiment, the step of analyzing the microbiome comprises nucleotide sequencing of bacterial nucleotide sequences present in the skin sample derived from the subject. In one embodiment, the nucleotide sequencing comprises sequencing of the gene encoding the 16S ribosomal RNA (16S rRNA).

In another embodiment, analyzing the skin microbiome in the sample comprises 16S rRNA gene sequencing or whole genome shotgun metagenomics. In one embodiment, the nucleotide sequencing comprises DNA sequencing.

In some embodiments, determining the abundance of any one of Corynebacteriaceae species, Staphylococcaceae species, or both, comprises detecting Corynebacteriaceae-specific region of the 16S ribosomal RNA gene, Staphylococcaceae-specific region of the 16S ribosomal RNA gene, or both.

In another embodiment, the step of analyzing the skin microbiome comprises identification of a specific nucleotide sequence via amplification, such as by PCR, of a gene fragment by gene-specific PCR primers.

In another embodiment, the step of analyzing the skin microbiome comprises an optical detection technique, an electrochemical detection technique, or a mass detection technique.

In embodiments, methods are provided which comprise, inter alia, collecting a biological sample from skin of a subject and analyzing the microbiome in the sample derived from the skin. Modern techniques for skin microbial analysis are known and within the understanding of the ordinarily skilled artisan and specific methods and techniques can be employed and adjusted to best suit the aims of the study.

In general, several procedural practices should be considered before the study begins, including avoiding contamination by environmental DNA, storage in warm conditions, and the exposure of the samples to researchers and clinicians. Further, precautions should be taken to ensure a sterile technique is utilized, and that bacterial DNA sequences (not only live bacterial organisms) are not introduced into the sample from sampling equipment, lab reagents, and clinicians.

To obtain samples, microorganisms from the skin can be collected by any suitable method known in the art, including, without limitation, swabbing, tape stripping, scraping or collecting biopsies using sterile techniques. Skin samples can be collected from any suitable location, including, without limitation, the nare, axillary vault, antecubital fossa, interdigital webspace, inguinal crease, gluteal crease, popliteal fossa, plantar heel, umbilicus, or a combination thereof. Appropriate and effective sample storage conditions should also be employed. If sterile sample collection is combined with effective storage conditions, an accurate representation of the skin microbiome should be maintained prior to DNA extraction and analysis. Once the samples are obtained and properly stored, DNA extractions can then be performed. Several different methods have been developed for the extraction of skin microbiome samples, including the REPLI-g Midi kit (Qiagen, Limberg, The Netherlands), Qiagen DNA Extraction Kit (Qiagen), and DNeasy DNA Extraction kit (Qiagen). In an effort to obtain the most accurate representation of the microbial diversity, studies have also explored different kit and non-kit based extraction methods, such as disruption of bacterial cell walls (e.g., bead beating or enzymatic lysis).

After DNA extraction, the specific target species or classes of microorganisms need to be identified to determine the most appropriate sequencing strategy. For example, bacterial communities can be assessed by amplifying a variable region of the conserved 16S ribosomal RNA gene, while fungal species and/or other eukaryote species can be targeted by applying 18S ribosomal RNA gene or the internal transcribed spacer.

While culturing methods can be used in detecting bacterial strains in accordance with embodiments described herein, targeted sequencing approaches do not require any culturing methods and hundreds of samples can be analyzed on a single sequencing run, providing an efficient and cost-effective means to examining microbial communities. Alternatively, shotgun sequencing can be performed, which will identify a subset of random DNA sequences from the sample. In either approach, sequencing technologies should also be taken into account. While Roche 454 or Illumina MiSeq can provide adequate sequencing coverage or depth for targeted amplicon sequencing, deeper coverage attainable through Illumina HiSeq or Pacific Biosciences technologies may be better suited for shotgun sequencing.

16S data processing—The 16S sequence data can be processed in accordance with any suitable techniques known to one of skill in the art, including as previously described (McDonald, et al., 2018). In one embodiment, processing can use a sequence variant method, such as Deblur v1.0.2, trimming to 125 nucleotides, to maximize the specificity of 16S data. Following processing by Deblur, previously recognized bloom sequences can be removed. The Deblur sOTUs can be inserted into the Greengenes 13_8 (19) 99% reference tree using SEPP. SEPP uses the simultaneous alignment and tree estimation strategy as previously described (Liu et al., 2009) to identify reasonable placements for sequence fragments within an existing phylogeny and alignment. Taxonomy can be assigned using an implementation of the RDP classifier as implemented in QIIME2. (McDonald, et al.).

Principal coordinates analysis can be undertaken in accordance with any suitable techniques known to one of skill in the art. In one embodiment, a distance matrix can be constructed using, for example, without limitation, the Bray Curtis dissimilarity index. In an embodiment, principal coordinates analyses can be implemented using, for example, without limitation, EMPeror software.

In some embodiments, the bacterial diversity of a skin microbiome which is indicative of increased likelihood to develop a metabolic syndrome or a condition associated therewith, is reduced by at least 5%, at least 10%, at least 20%, at least 35%, at least 50%, at least 65%, at least 80%, at least 90%, at least 95%, or at least 99%, compared to control, or any value and range therebetween. Each possibility represents a separate embodiment of the invention. In some embodiments, the bacterial diversity of a skin microbiome which is indicative of increased likelihood to develop a metabolic syndrome or a condition associated therewith, is reduced by 1-25%, 5-45%, 10-75%, 15-85%, 5-35%, 40-95%, 25-100%, 30-65%, 45-85%, or 60-99%. Each possibility represents a separate embodiment of the invention.

In some embodiments, a skin microbiome indicative of increased likelihood to develop a metabolic syndrome or a condition associated therewith comprises a Corynebacteriaceae species having an abundance of at least 5%, at least 10%, at least 15%, at least 25%, at least 30%, at least 45%, at least 55%, at least 75%, at least 85%, at least 95%, or at least 99%, within the skin microbiome, or any value and range therebetween. Each possibility represents a separate embodiment of the invention. In some embodiments, a skin microbiome indicative of increased likelihood to develop a metabolic syndrome or a condition associated therewith comprises a Corynebacteriaceae species having an abundance of 1-25%, 5-35%, 15-55%, 10-85%, 20-99%, 1-50%, 20-75%, 30-65%, 75-95%, 60-100%, within the skin microbiome. Each possibility represents a separate embodiment of the invention.

In some embodiments, a skin microbiome indicative of increased likelihood to develop a metabolic syndrome or a condition associated therewith comprises a Corynebacteriaceae species and a Staphylococcaceae species in an abundance ratio ranging from 3:1 to 9:1. In some embodiments, a skin microbiome indicative of increased likelihood to develop a metabolic syndrome or a condition associated therewith comprises a Corynebacteriaceae species and a Staphylococcaceae species in an abundance ratio ranging from 3.2:1 to 9:1. In some embodiments, ratio ranging from 3.2:1 to 9:1 comprises: 3.2:1 to 8:1, 3.2:1 to 7:1, 3.2:1 to 6:1, 3.2:1 to 5:1, 3.2:1 to 4:1, 4:1 to 9:1, 5:1 to 8:1, 6:1 to 9:1, or 7:1 to 12:1. Each possibility represents a separate embodiment of the invention.

In some embodiments, the subject has a body mass index (BMI) value ranging from 19 to 29. In some embodiments, the subject has a BMI value ranging from 19 to 26. In some embodiments, a BMI value ranging from 19 to 29 comprises a BMI value of 19 to 28, 19 to 27, 20 to 26, 19 to 25, 25 to 29, 26 to 29, 27 to 29, 28 to 30. Each possibility represents a separate embodiment of the invention.

BMI calculation would be apparent to one of ordinary skill in the art.

In some embodiments, a skin microbiome indicative of increased likelihood to develop a metabolic syndrome or a condition associated therewith comprises a Corynebacteriaceae species having an abundance of at least 5%, at least 10%, at least 15%, at least 25%, at least 30%, at least 45%, at least 55%, at least 75%, at least 85%, at least 95%, or at least 99%, within the skin microbiome of a subject having a BMI value ranging from 19 to 29, 19 to 27, 20 to 26, 19 to 25, 25 to 29, 26 to 29, 27 to 29, 28 to 30.

In some embodiments, a subject having a skin microbiome indicative of increased likelihood to develop a metabolic syndrome or a condition associated therewith comprises a Corynebacteriaceae species having an abundance of at least 15% within the skin microbiome and has a BMI value of 19 to 26.

As used herein the term “metabolic syndrome” refers to any disease, disorder, or condition characterized by any one of the following: excess abdominal fat, hypertension, abnormal fasting plasma glucose level or insulin resistance, high triglyceride levels, and low high-density lipoprotein (HDL) cholesterol level. The metabolic syndrome which can be predicted or treated according to the present invention include, but is not limited to, fatty liver, obesity, pre-diabetes, diabetes, hyperglycemia, diabetic dyslipidemia, hyperlipidemia, hypertriglyceridemia, hypercholesterolemia, hyperinsulinemia, insulin-resistance or insulin-resistance related.

In one embodiment, the metabolic syndrome is a fatty liver disease. The term “fatty liver disease” (FLD) refers to a disease or a pathological condition caused by, at least in part, abnormal hepatic lipid deposits. Fatty liver disease includes, e.g., alcoholic fatty liver disease, nonalcoholic fatty liver disease, and acute fatty liver of pregnancy. Fatty liver disease may be, e.g., macrovesicular steatosis or microvesicular steatosis. In one embodiment, the metabolic syndrome is nonalcoholic steatohepatitis (i.e., fatty liver). In one embodiment, the metabolic syndrome comprises hepatotoxicity, nephrotoxicity or both, wherein the hepatotoxicity, nephrotoxicity, or both are fat-induced. As used herein, fat induced-hepatotoxicity, and fat-induced nephrotoxicity, refer to any disease or condition encompassing increased toxicity to cells of the liver, kidney, or both which is attributed to the accumulation of fat, triglycerides, free fatty acid, lipid droplet, or any equivalent thereof, which negatively impacts the intoxicated cell. Intoxicated cells, for example, undergo necrosis, lose membrane integrity, undergo cell lysis, cease growing or cell division, actively induce apoptosis.

In one embodiment, the metabolic syndrome is a fatty kidney disease (FKD). The term “fatty kidney disease” encompasses any disease or a pathological condition comprising ectopic lipid deposits in the kidney.

In some embodiments, the metabolic syndrome is diabetes. In some embodiments, diabetes is type 2 diabetes (T2DM). In some embodiments, the metabolic syndrome is an insulin resistance-related disease. In some embodiments, the metabolic syndrome is an insulin resistance-related-T2DM. In some embodiments, the metabolic syndrome is an insulin resistance-dependent-T2DM. In some embodiments, the metabolic syndrome is a muscular disease. In some embodiments, the metabolic syndrome is a metabolic muscular disease. In some embodiments, the metabolic syndrome is a cardiac muscle metabolic disease (i.e., cardiometabolic disease). In some embodiments, the metabolic syndrome is a skeletal muscle metabolic disease.

In some embodiments, a subject having increased likelihood to develop a metabolic syndrome or a condition associated therewith, is characterized by having or by being afflicted with: abnormal fat metabolism, alcoholism, advanced age (e.g., greater than 40, 50, 60, or 70 years of age), celiac disease, diabetes mellitus (e.g., type II diabetes mellitus), dyslipidemia, exposure to industrial solvents, galactosemia, glycogen storage diseases, homocystinuria, hyperferritinemia, hyperinsulinemia, hyperlipidemia, hypertension, hypertriglyceridemia, hyperuricemia, hypoxia, impaired fasting glycemia, inborn metabolic disorders (e.g., related to galactose, glycogen, homocysteine, or tyrosine metabolism), insulin resistance, iron overload, jejunal bypass surgery, low levels of high-density lipoprotein, Madelung's lipomatosis, malnutrition, Mauriac syndrome, metabolic syndrome, mitochondrial dysfunction, mitochondrial injury, mitochondrialopathies, niacin deficiency, Niemann-Pick disease, obesity (especially visceral adiposity or central obesity), overnutrition, pantothenic acid deficiency, peroxisomal diseases, polycystic ovarian syndrome, pregnancy, rapid weight loss, riboflavin deficiency, sleep apnea, starvation, tyrosemia, Weber-Christian disease, or Wilson's disease may have, or be at increased risk of developing, a disorder associated with hepatic lipid deposits. In some embodiments, a subject having increased likelihood to develop a metabolic syndrome, or a condition associated therewith, is treated with certain medications, such as, e.g., amiodarone, corticosteroids, estrogens (e.g., synthetic estrogens), maleate, methotrexate, perhexyline, salicylate, tamoxifen, tetracycline, and valproic acid.

In some embodiments, the metabolic syndrome or a condition associated therewith is selected from: obesity, pre-diabetes, diabetes, hyperglycemia, diabetic dyslipidemia, hyperlipidemia, hypertriglyceridemia, hypercholesterolemia, hyperinsulinemia, and insulin-resistance or insulin-resistance related.

In some embodiments, the agent or composition comprising thereof is modifying the skin microbiome.

As used herein, the term “appetite suppressant agent” refers to any compound capable of reducing appetite, and thereby reduces food consumption and/or promotes weight loss. Types of appetite suppressants are common and would be apparent to a skilled artisan. In some embodiments, an appetite suppressant comprises any one of: conjugated linoleic acid (CLA), Synephrine, Garcinia cambogia, glucomannan, Hoodia gordonii, green coffee bean extract, Guarana, Acacia fiber, Saffron extract, Guar gum, Forskolin (or Coleus forskohii extract), Chromium picolinate, Fenugreek, Gymnema sylvestre, 5-hydroxytryptophan (or Griffonia simlicifolia extract), Caralluma fimbriata, and Yerba mate.

As used herein, the term “probiotic agent” refers to any microorganism improving health benefits on a subject consuming the same. In some embodiments, a probiotic is defined as a living microorganism that, when administered in adequate amounts, confers a health benefit on the host. In some embodiments, the probiotic agent improves the subject's natural flora, or microbiome. In some embodiments, the probiotic agent restores the subject's natural flora or microbiome. Types of probiotics are common and would be apparent to a skilled artisan. In some embodiments, a probiotic is selected from: Lactobacillus (e.g., L. rhamnosus, L. acidophilus, L. plantarum, L. paracasei, L. bulgaricus), Saccharomyces boulardii, Bifidobacterium (e.g., B. breve, B. longum, B. infantis), and Streptococcus thermophilus.

As used herein, the term “prebiotic agent” refers to any dietary compound which induces or sustains activity, growth, or both, of beneficiary bacteria or fungi. Types of prebiotics are common and would be apparent to a skilled artisan. In some embodiments, a prebiotic is selected from raw, dry, or both, of any one of: chicory root, Jerusalem artichoke, dandelion greens, garlic, leek, onion, asparagus, wheat bran, whole wheat flour, banana, walnuts, oats, unrefined barely, yacon (also known as ‘vegetarian bacon’), whole grain cereal. In some embodiments, a prebiotic comprises a prebiotic fiber. In some embodiments, the prebiotic fiber is selected from: beta glucan, pectin, gum, inulin, oligofructose, starch (e.g., a resistant-starch).

As used herein, the term “anti-inflammatory drug” refers to any compound capable of reducing or inhibiting an inflammatory response.

In some embodiments, an anti-inflammatory drug is any one of: an antibiotic, a tumor necrosis factor α (TNFα) inhibitor/blocker, a non-steroidal anti-inflammatory drug (NSAID), and any combination thereof.

In some embodiments, a TNFα inhibitor/blocker is selected from: infliximab, adalimumab, certolizumab pegol, golimumab, and etanercept. In some embodiments, a TNFα inhibitor/blocker is selected from: thalidomide, xanthine, dexamethasone, or tacrolimus.

In some embodiments, a NSAID is selected from: aspirin, celecoxib, diclofenac, diflunisal, etodolac, ibuprofen, indomethacin, or acetaminophen.

In another embodiment, the composition comprises a topical solution. In some embodiments, a composition comprising a topical solution is administered topically to body surfaces and is thus formulated in a form suitable for topical administration. Suitable topical formulations include gels, ointments, creams, lotions, drops and the like. For topical administration, the therapeutic agent is prepared and applied as a solution, suspension, or emulsion in a physiologically acceptable diluent with or without a pharmaceutical carrier.

In one embodiment, the treatment for modifying the skin microbiome comprises administering an emollient to the subject. In another embodiment, the treatment for modifying the skin microbiome comprises administering a food product to the subject. In one embodiment, the treatment for modifying the skin microbiome comprises administering an emollient and a food product to the subject. In one embodiment, the treatment for modifying the skin microbiome comprises a dietary supplement. In one embodiment, the food product is any one of: snack bar, cookie, muffin, cake, bread, cereal, juice, yogurt, milk, dairy product, infant formula, and the like. In one embodiment, the food product for modifying the skin microbiome or the dietary supplement comprises one or more probiotics, one or more prebiotics, or any combination thereof.

In another embodiment, the present invention provides a method of altering or modifying the skin microbiome in a subject having or prone to developing a metabolic syndrome or a condition associated therewith, comprising the step of administering to the subject a therapeutically effective amount of a composition comprising an agent capable of modifying the skin microbiome.

In some embodiments, the composition is a pharmaceutical composition, a nutraceutical composition, or a combination thereof.

In one embodiment, the composition modifies or alters the bacterial diversity of the skin microbiome. As used herein, the terms “modify” or “modifying” comprise increasing or decreasing. The terms “modify” and “alter” are used herein interchangeably.

In some embodiments the composition decreases Corynebacteriaceae abundance, colonization, or both. In some embodiments the composition decreases Corynebacterium abundance, colonization, or both. In some embodiments the composition decreases S. aureus abundance, colonization, or both. In some embodiments the composition increases the abundance, colonization, or both, of S. epidermidis, S. homonis, or both. In some embodiments the composition decreases the abundance, colonization, or both, of Corynebacteriaceae and S. aureus, and increases the abundance, colonization, or both, of S. epidermidis, S. homonis, or both.

In another embodiment, a composition as provided herein can be a controlled-release composition, i.e. composition in which the therapeutic agent is released over a period of time after administration. Controlled- or sustained-release compositions include formulation in lipophilic depots (e.g. fatty acids, waxes, oils). In another embodiment, the composition is an immediate-release composition, i.e. a composition in which all of the therapeutic agent is released immediately after administration.

In one embodiment, the composition is formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

It may be desirable to locally administer a composition in the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material. According to some embodiments, administration can be by direct injection e.g., via a syringe.

Effective dose of the composition for treatment of a condition or a disease vary depending upon many different factors, including means of administration, target site, physiological state of the patient, whether the patient is human or an animal, other medications administered, and whether treatment is prophylactic or therapeutic. Usually, the patient is a human, but non-human mammals including transgenic mammals can also be treated. Treatment dosages may be titrated using routine methods known to those of skill in the art to optimize safety and efficacy. The composition thus may include a “therapeutically effective amount.” A “therapeutically effective amount” refers to an amount effective, at dosages and for periods of time necessary, to achieve the desired therapeutic result. A therapeutically effective amount of a molecule may vary according to factors such as the disease state, age, sex, and weight of the individual, and the ability of the molecule to elicit a desired response in the individual. A therapeutically effective amount is also one in which any toxic or detrimental effects of the molecule are outweighed by the therapeutically beneficial effects.

Furthermore, a skilled artisan would appreciate that the term “therapeutically effective amount” may encompass total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

The amount of a compound that will be effective in the treatment of a particular disorder or condition as disclosed herein, including a metabolic syndrome or a condition associated therewith, also will depend on the nature of the syndrome or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. In one embodiment, the dosage will be within the range of 0.01-1,000 mg/kg of body weight per day. In another embodiment, the dosage will be within the range of 0.1 mg/kg per day to 100 mg/kg per day. In another embodiment, the dosage will be within the range of 1 mg/kg per day to 10 mg/kg per day. Effective doses may be extrapolated from dose-response curves derived from in vitro or animal model test bioassays or systems.

For therapeutic purposes, the active compound is ordinarily combined with one or more adjuvants appropriate to the indicated route of administration.

A pharmaceutical composition as disclosed herein optionally comprises an additional agent selected from any pharmaceutically acceptable carrier, adjuvant, and vehicle.

In some embodiments, a method for treating a metabolic syndrome or a condition associated therewith further comprises modifying the diet of said subject, wherein modifying is changing in a personalized manner. The term “personalized” refers to the that the diet is changed in a manner so as to provide specific needs of the subject, based on, for example, the subject's lipid profile, gut microbiome, sugar levels, blood pressure, physical activity levels, any combination thereof, and others.

In some embodiments, the modified diet comprises any diet selected from: a diet low on calories, a diet low on fat content, a diet low on carbohydrates content, and any combination thereof.

In some embodiments, the modified diet comprises providing a diet low on calories.

In some embodiments, modifying is alternating the timing of food consumption. In some embodiments, alternating the timing of food consumption comprises: modifying the hours wherein food is consumed (e.g., earlier or later throughout a determined period of time, for example a day), modifying the gap or interval between meals or food consumption events, modifying the number of meals or food consumption events per a determined period of time, e.g., 24 hours or a day, and any combination thereof.

Types of diet, and personalization of same so as to meet the subject's specific physical needs, would be apparent to one of ordinary skill in the art of dietetics.

In some embodiments, the method is for preventing a metabolic syndrome or a condition associated therewith. In some embodiments, preventing comprises any one of: reducing the severity, delaying the onset, reducing the cumulative incidence, and any combination thereof, of a metabolic syndrome or a condition associated therewith.

As used herein, the terms “subject” or “individual” or “animal” or “patient” or “mammal,” refers to any subject, particularly a mammalian subject, for whom therapy is desired, for example, a human.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include” and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Unless specifically stated or obvious from context, as used herein, the term “or” is understood to be inclusive.

Throughout this specification and claims, the word “comprise” or variations such as “comprises” or “comprising,” indicate the inclusion of any recited integer or group of integers but not the exclusion of any other integer or group of integers.

As used herein, the term “consists essentially of”, or variations such as “consist essentially of” or “consisting essentially of” as used throughout the specification and claims, indicate the inclusion of any recited integer or group of integers, and the optional inclusion of any recited integer or group of integers that do not materially change the basic or novel properties of the specified method, structure or composition.

As used herein, the terms “comprises”, “comprising”, “containing”, “having” and the like can mean “includes”, “including”, and the like; “consisting essentially of or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. In one embodiment, the terms “comprises”, “comprising”, “having” are/is interchangeable with “consisting”.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

### Material and Methods

### The American Gut Project (AGP)

Sample processing, sequencing, and core amplicon data analysis were performed by the American Gut Project (www.americangut.org) and all amplicon sequence data and metadata have been made public through the AGP data portal (qiita.ucsd.edu; study ID 10317). Briefly, AGP sample collection and analyses was approved by the Institutional Review Boards of the University of Colorado Boulder and of the University of California, San Diego. Participants signed up online and self-reported metadata. Samples were shipped to the Knight lab and processed using the Earth Microbiome Project protocols. 16S rRNA data was processed using Deblur v1.0.2 and bloom sequences were removed. Greengenes 13_8, SEPP, RDP classifier and QIIME2 were used for alignment, tree building, and taxonomy assignment.

### Data Accession & Sample Selection

Data for these analyses were obtained from the American Gut ftp site (ftp://ftp.microbio.me/AmericanGut/). The inventors included all samples where Body mass index (BMI) data was made available, height and weight were provided. The inventors subsequently filtered the data to include individuals whose BMI was between 15 and 45.

### Alpha Diversity, Beta-Diversity & Taxonomic Relative Abundance

Phylogenetic diversity (PD)-whole-tree, Shannon, Chao-1 and observed OTU diversity metrics were calculated with QIIME1 (alpha_diversity.py). Beta-diversity metrics were calculated and plotted using Bray-Curtis dissimilarity in QIIME1 (beta_diversity_throughplots.py) and significance between sample categories was tested with PERMANOVA in QIIME1 (compare_categories.py). Taxonomic relative abundance tables were generated at all taxonomic levels in QIIME1 (summarize_taxa.py). Alpha diversity plots and taxonomic relative abundance plots were plotted using GraphPad Prism 7.02. Alpha diversity and taxonomic relative abundance were compared with the Wilcoxon rank sum test in R (pairwise.wilcox.test). Multiple hypothesis correction was carried out using the false discovery rate.

### Linear Discriminant Analysis Effect Size Analysis (LEfSe)

The inventors used the online Galaxy interface (https://huttenhower.sph.harvard.edu/galaxy/root) to identify differentially abundant genera by BMI category with LEfSe. BMI category was assigned as the comparison class, and overweight and obese individuals were clustered into the same category (Overweight/Obese). Default LEfSe settings were used, including 0.05 alpha value for factorial Kruskal-Wallis test, 2.0 logarithmic linear discriminant analysis (LDA) score for discriminative features, and an all-against-all strategy for multi-class analysis.

### Example 1

### Distinct Microbiomes by Body Site

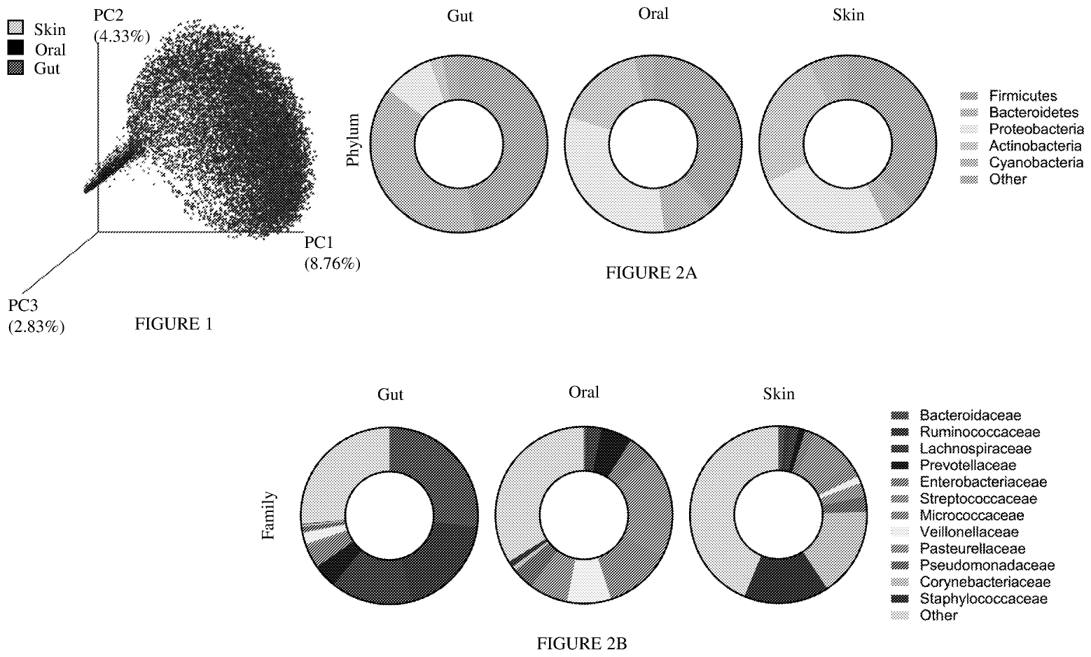

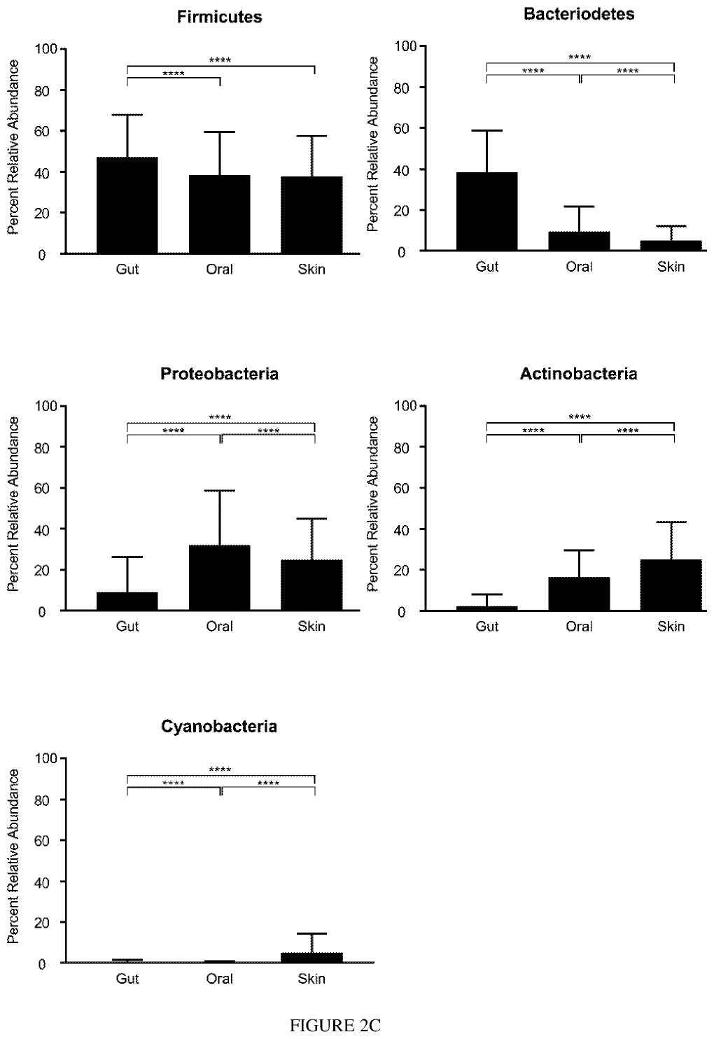

The inventors analyzed 16,353 samples with BMI data, of which 14,287 samples were fecal (87.4%), 1,071 were oral (6.5%) and 995 were from skin sites (6.1%). The inventors observed significant partitioning using PERMANOVA between fecal, oral and skin microbiomes (FIG. 1), consistent with previous reports of the human microbiome. All body sites were dominated by the bacterial phyla Firmicutes, Bacteroidetes, Proteobacteria and Actinobacteria. Cyanobacteria were also represented on skin samples (FIG. 2A). While all body sites were dominated by Firmicutes, Bacteroidetes, Proteobacteria and Actinobacteria, their percent relative abundance on body sites were significantly different, with the exception of Firmicutes when comparing oral and skin sites (FIG. 2C). Dominant bacterial families in gut communities comprised of Bacteroidaceae and Ruminococcaceae, dominant bacterial families in oral communities comprised of Streptococcaceae, Micrococcaceae, Veillonellaceae and Pasteurellaceae, and dominant bacterial families in skin communities comprised of Enterobacteriaceae, Corynebacteriaceae, and Staphylococcaceae. These findings support previously established characterizations of the gut, oral and skin microbiomes.

### Example 2

### Gut and Skin Bacterial Community Diversity is Shaped by BMI

The inventors clustered samples into four BMI categories; underweight (BMI 15-18.5), normal weight (BMI 18.5-25), overweight (BMI 25-30) and obese (BMI 30-45). Sample distribution by site and BMI is illustrated in Table 1.

TABLE 1
AGP Sample Distribution by Body Site and BMI

| | Fecal | Oral | Skin |
|---|---|---|---|
| Underweight (BMI 15- | 1,359 (9.5%) | 88 (8.2%) | 80 (8.1%) |
| 18.5) | | | |
| Normal weight (BMI 18.5- | 8,092 (56.7%) | 621 (58.1%) | 580 (58.5%) |
| 25) | | | |
| Overweight (BMI 25-30) | 3,425 (24.0%) | 124 (22.0%) | 81 (25.2%) |
| Obese (BMI 30-45) | 1,408 (9.9%) | 124 (11.6%) | 81 (8.2%) |

A large majority of AGP samples were from the gut (14,284/16,063), yet oral and skin datasets were much larger than in most previously published microbiome research endeavors. Additionally, over half of samples in all body sites represented individuals with normal BMI, roughly one quarter were overweight and underweight and obese individuals accounted for about one tenth of samples respectively. BMI distribution is similar between fecal, oral and skin samples.

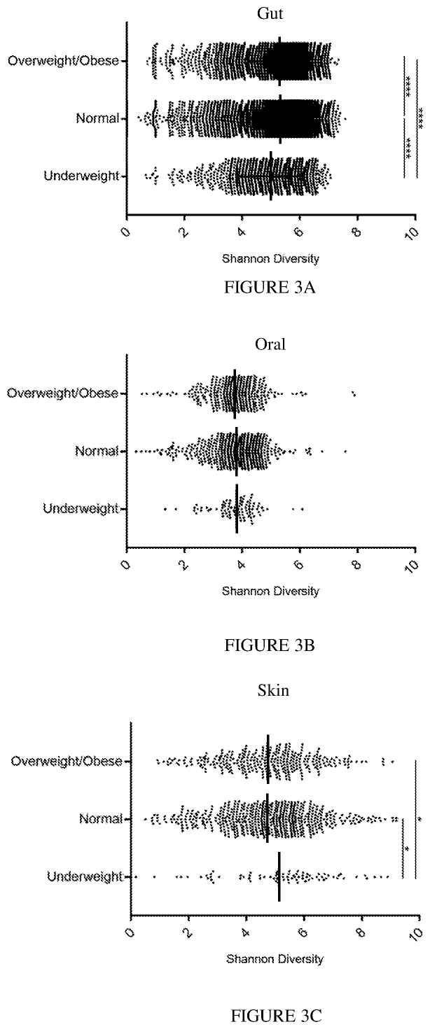

The inventors compared community diversity between BMI categories using the Shannon diversity index, which accounts for both species richness and evenness. Shannon diversity of gut microbial communities was significantly different between overweight/obese, normal and underweight individuals, consistent with previous reports of the gut microbiome and BMI (FIG. 3A). There were no significant differences in bacterial community diversity in the oral microbiome samples. Interestingly, skin microbiome communities were significantly different between overweight/obese individuals and underweights individuals (p=0.02, FIG. 3B), as well as between normal weight individuals and underweight individuals (p=0.02, FIG. 3C). There was no significance observed between normal and overweight/obese individuals on skin communities.

### Example 3

### BMI Shapes the Skin Microbiome

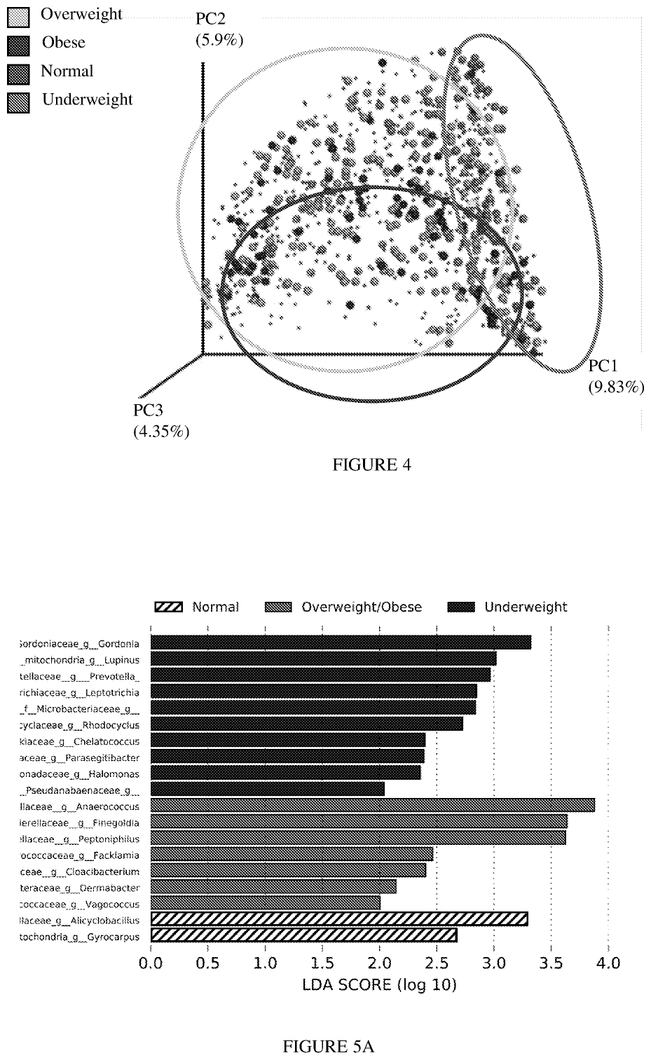

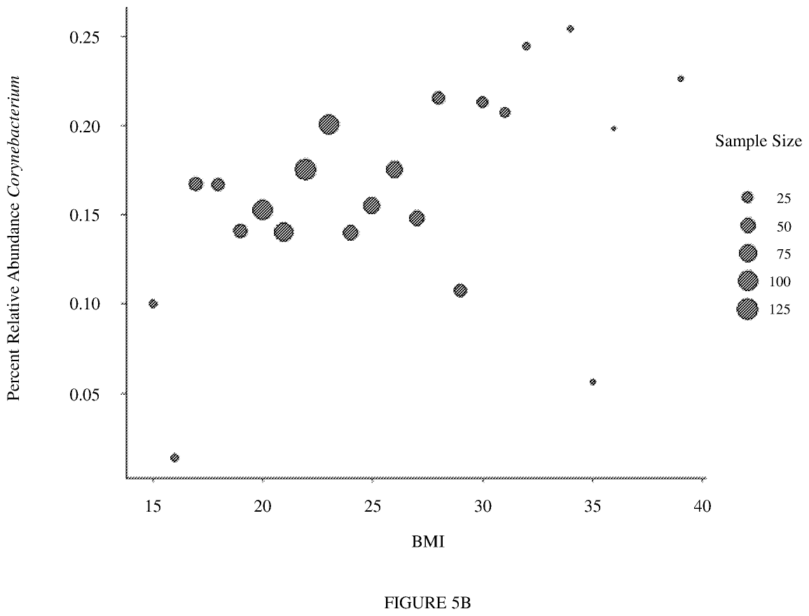

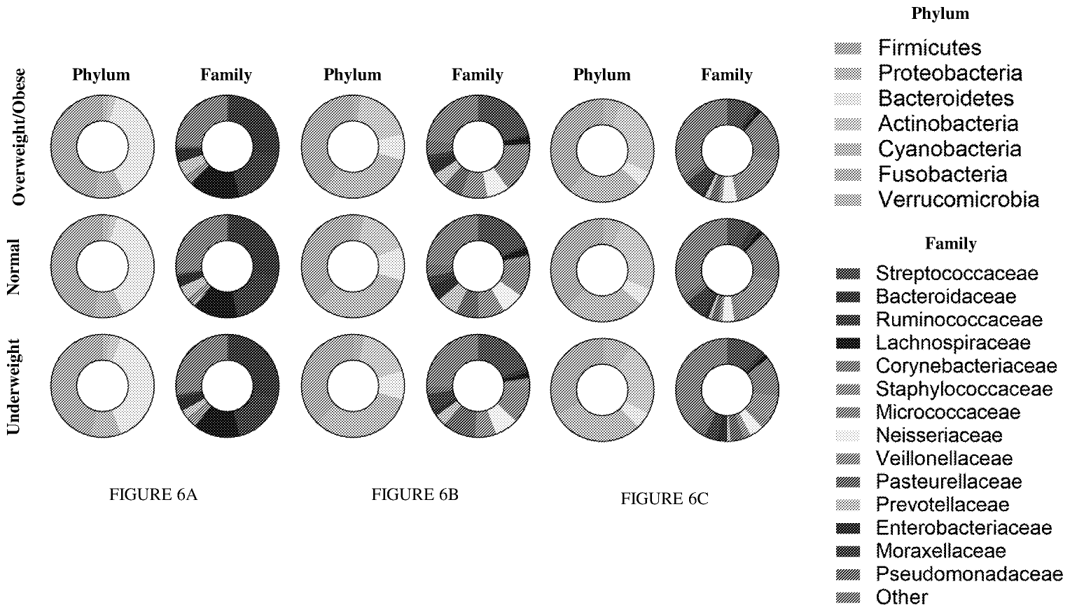

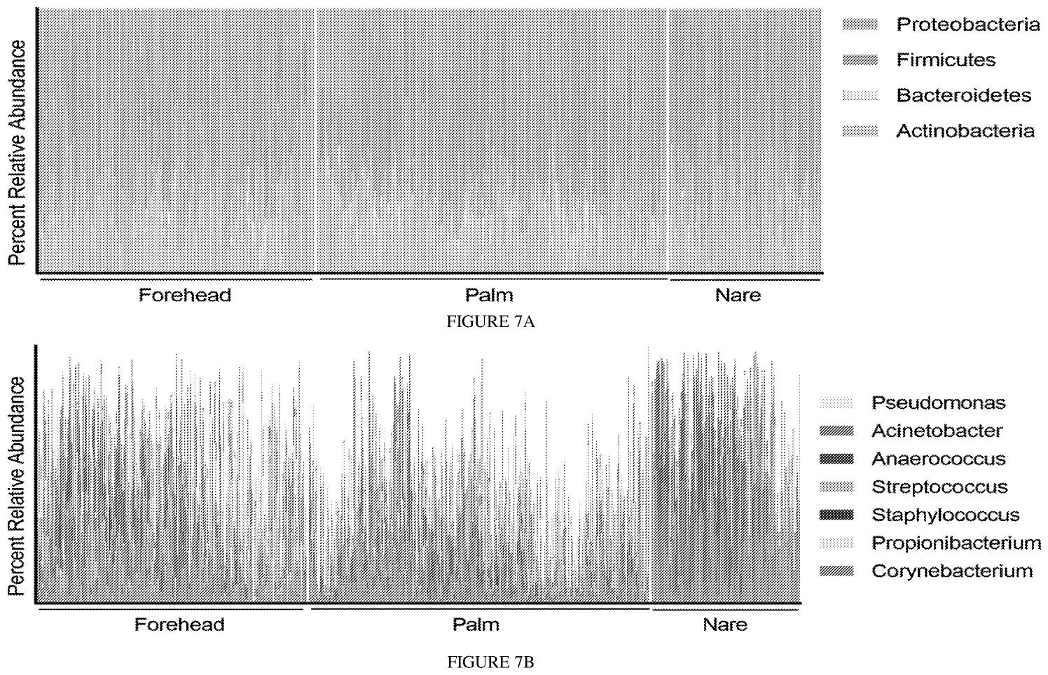

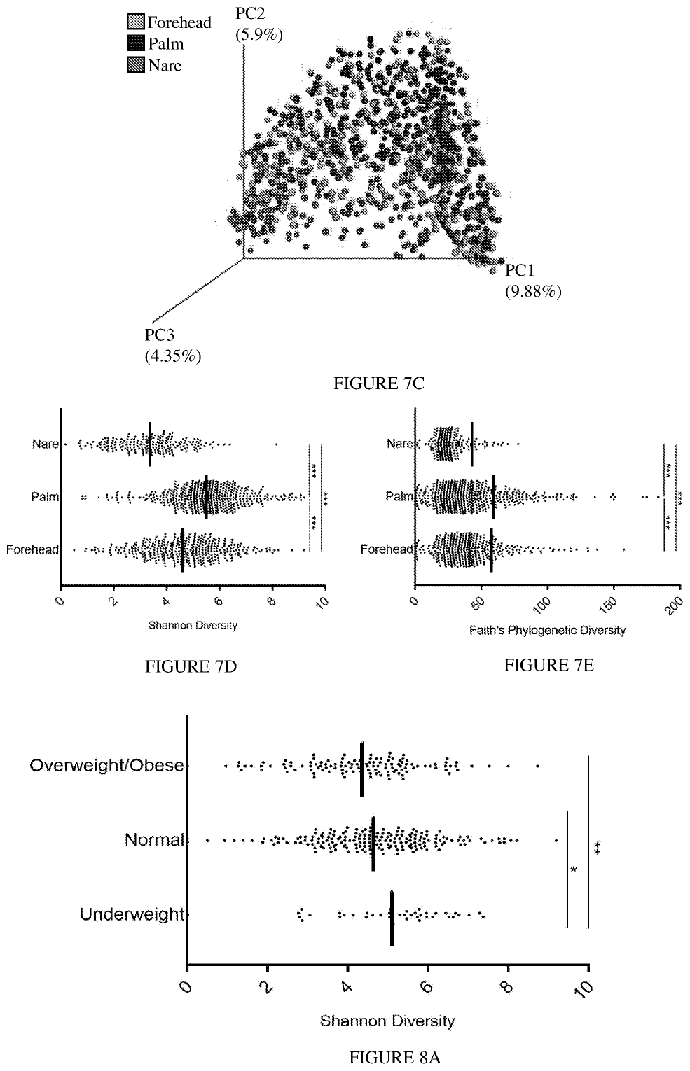

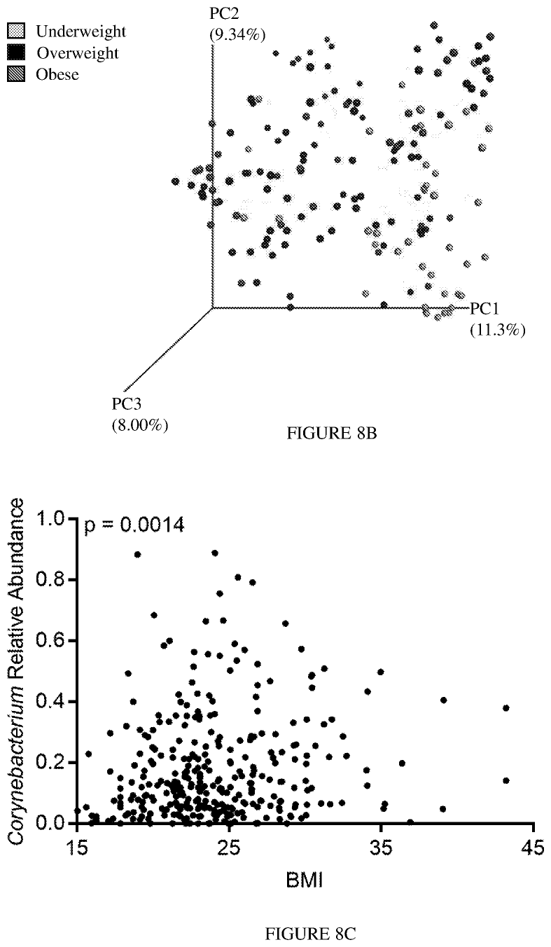

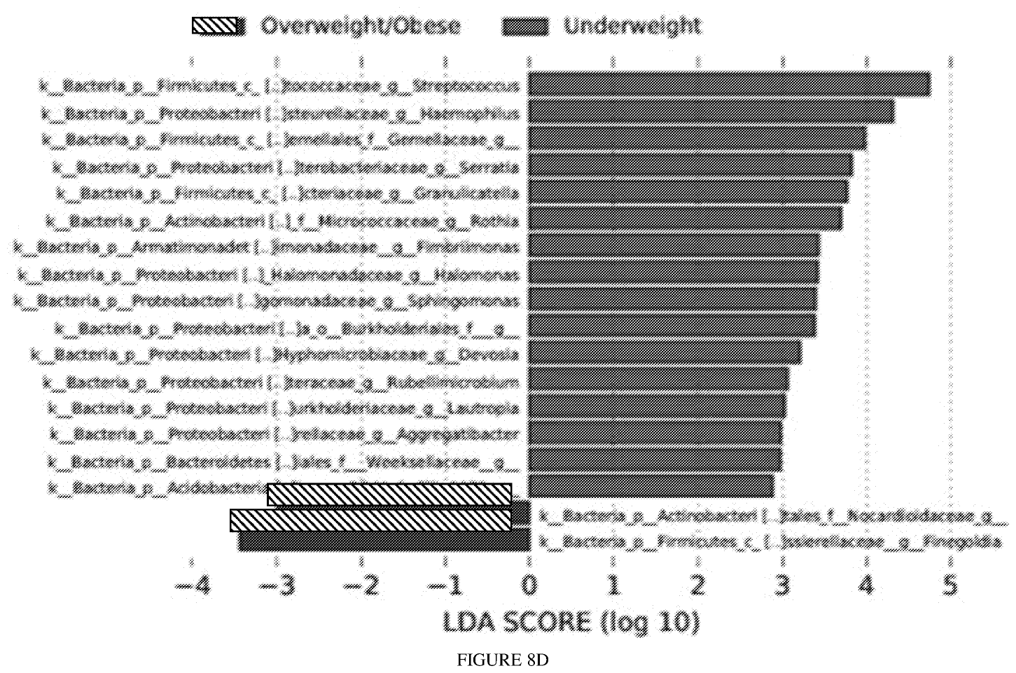

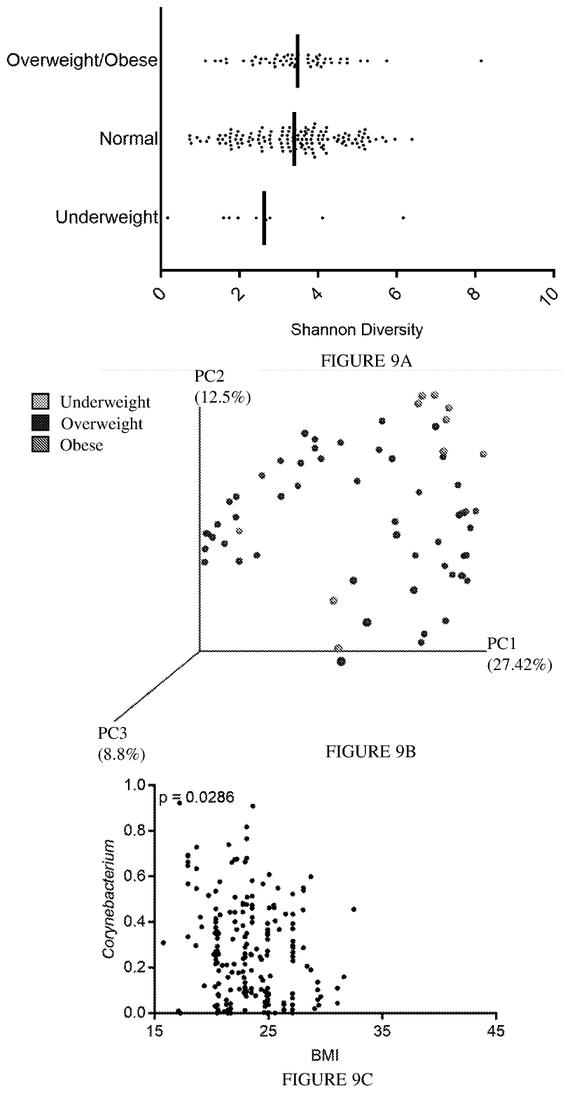

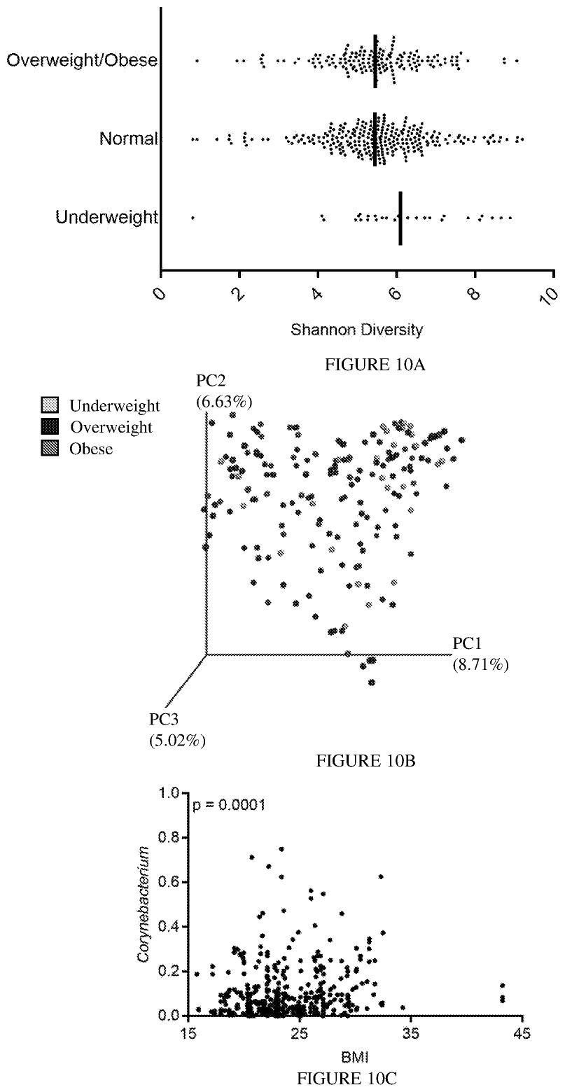

Following the above-mentioned indication that community diversity is significantly altered based on BMI category, the inventors further explored the skin microbiome and its relationship to BMI. Significant partitioning was observed between BMI categories using PERMANOVA (p=0.001, FIG. 4). The inventors compared Bray-Curtis distances between all Obese vs. Normal and Normal vs. Underweight samples, which was highly significant (p>0.0001). The inventors then used the LEfSe algorithm to identify taxonomic differences of skin microbial communities associated with BMI categories (FIG. 5A). The inventors set the logarithmic LDA score cutoff to 2.0 to identify differentially abundant bacteria by BMI category. LEfSe results indicated ten different microbial genera enriched in underweight individuals, including Gordonia, Lupinus, and Prevotella. An additional seven genera were enriched in overweight/obese individuals, including Anaerococcus, Finegoldia and Peptoniphilus. Two genera were enriched in normal weight skin; Alicyclobacilus and Gyrocarpus.

Given the recently uncovered association between obesity in mice and cutaneous Corynebacterium colonization, the inventors plotted taxonomic relative abundance of Corynbacterium versus BMI levels (FIG. 5B). Corynebacterium taxonomic relative abundance was significantly correlated with BMI (p=0.0002), further expanding the connection between this microorganism and obesity.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1-11. (canceled)

12. A method for treating or preventing a metabolic syndrome or a condition associated therewith in a subject in need thereof, the method comprising:
a. determining any one of: abundance of Corynebacteriaceae species, ratio of Corynebacteriaceae to Staphylococcaceae, and both, in a facial skin of said subject, wherein any one of: (i) a reduction of at least 5% in bacterial diversity in a facial skin of said subject compared to control, (ii) an abundance of at least 15% of a Corynebacteriaceae species in a facial skin of said subject, (iii) a Corynebacteriaceae to Staphylococcaceae abundance ratio ranging from 3.2:1 to 9:1 in a facial skin of said subject; or a combination of (i) to (iii), is indicative of said subject being at increased risk of developing a metabolic syndrome or a condition associated therewith; and
b. administering to a subject determined as being at an increased risk, a therapeutically effective amount of a pharmaceutical or a nutraceutical composition comprising an agent selected from the group consisting of: an appetite suppressant, a probiotic agent, a prebiotic agent, an anti-inflammatory drug, a topical solution, and any combination thereof,
thereby treating or preventing a metabolic syndrome or a condition associated therewith in a subject.

13. The method of claim 12, further comprising modifying the diet of said subject.

14. The method of claim 13, wherein said modifying is providing any one of: a diet low on calories, a diet low on fat content, a diet low on carbohydrates content, and any combination thereof.

15. The method of claim 13, wherein said modifying is providing a diet low on calories.

16. The method of claim 13, wherein said modifying is alternating the timing of food consumption.

17. The method of claim 12, wherein said preventing comprises any one of: reducing the severity, delaying the onset, reducing the cumulative incidence, and any combination thereof, of said metabolic syndrome or said condition associated therewith.

18. The method of claim 12, wherein said metabolic syndrome or said condition associated therewith is selected from the group consisting of: obesity, pre-diabetes, diabetes, hyperglycemia, diabetic dyslipidemia, hyperlipidemia, hypertriglyceridemia, hypercholesterolemia, hyperinsulinemia, and insulin-resistance or insulin-resistance related.

19. The method of claim 12, wherein said subject has a body mass index (BMI) value ranging from 19 to 29.

20. The method of claim 12, wherein said subject has a BMI value ranging from 19 to 26.

21. The method of claim 12, wherein said facial skin is the skin of any one of: the forehead, the glabella, or a combination thereof.

22. The method of claim 12, wherein said determining is determining in a sample derived from said subject.

23. The method of claim 22, further comprising providing a sample from said subject and performing said determining in said sample.
